(19) United States
(12) Patent Application Publication
(10) Pub. No.: US 2015/0140109 A1
(43) Pub. Date: May 21, 2015

(54) DOCETAXEL-BASED PROLONGED-RELEASE CANCER TREATMENT DRUG

(71) Applicants: OY FILANA LTD, Aura (FI); UNICHEMPHARM LTD, Edinburgh, Scotland (GB)

(72) Inventors: Evgenij Severin, Moscow (RU); Irina Zykova, Moscow (RU); Victor Gulenko, Dmitrov (RU); Maksim Iurchenko, Kiev (UA)

(73) Assignees: UNICHEMPHARM LTD, Edinburgh, Scotland (GB); OY FILANA LTD, Aura (FI)

(21) Appl. No.: 14/401,744

(22) PCT Filed: May 17, 2013
(86) PCT No.: PCT/FI2013/050538

§ 371 Date: Nov. 17, 2014

Related U.S. Application Data
(60) Provisional application No. 61/648,729, filed on May 18, 2012.

(30) Foreign Application Priority Data
May 18, 2012 (FI) 20125532

(51) Int. Cl. A61K 31/337; A61K 9/14; A61K 47/34; A61K 47/42; A61K 47/26

(52) U.S. Cl. CPC A61K 31/337; A61K 47/42; A61K 47/26; A61K 47/34; A61K 9/146; A61K 9/145
USPC 424/499; 514/449

(57) ABSTRACT

The present invention relates to the field of pharmacology and medicine, specifically to a new generation of anticancer drugs with the cytostatic action based on docetaxel. In the drug composition of the invention docetaxel is included in biodigestible stable nanoparticles. The nanoparticles comprise of docetaxel, PLGA, serum albumin and D-mannitol The developed drug is proposed to be manufactured as enterosoluble tablets, capsules granules, powder, or in any other peroral form.

### FIELD OF THE INVENTION

The present invention relates to the field of pharmacology and medicine, specifically to a new generation of anticancer drugs with the cytostatic action based on docetaxel. In the drug composition of the invention docetaxel is included in biodigestible stable nanoparticles.

### BACKGROUND OF THE INVENTION

According to the World Health Organization (WHO) figures, after 20 years the number of lethal cases from cancer could increase to 17 million per year. In its declaration, WHO stated that cancer, at the moment, is the most killing disease, in which connection this trend is the same for the developed countries as for the third world countries. In 2009 oncologic diseases were diagnosed in 12 million people, and one fourth of these people have no chance to survive more than 3 years after the diagnosis. Without creating a new concept of treatment and prevention of oncologic diseases, cancer mortality may increase in the next 20 years by 250%.

In Russia, in 1999 among cases of malignant neoplasms of men, colorectal cancer is amounted to 8.7%, firmly occupying the third place after lung cancer (26.5%) and stomach cancer (14.2%). Among women with cancer, the respective figure is 11.1%, after breast cancer (18.3%) and skin cancer (13.7%). The enumerated statistics indicate that the development of highly efficient drugs for the oncologic diseases treatment is a vital problem for Russia.

Docetaxel is one of the modern cytotoxic drugs of plant origin from the group of taxanes. Docetaxel is widely used together with other antineoplastic, hormonal and antihormonal drugs in case of metastatic breast cancer, metastatic non-small-cell lung cancer, metastatic ovarian cancer, metastatic head and neck squamous cell carcinoma, metastatic hormone refractory prostate cancer and metastatic stomach cancer [1-3]. A particularly high efficacy of the drug is noted in metastatic disease of hepatic glands and lungs.

The action of docetaxel is associated with the damage of the micro glandular network in the cells at the stage of the indirect nuclear division and in the interphase. Docetaxel is connected with the floating tubulin, stimulates the collection of tubulin into stable microtubules and prevents their collapse. Consequently, the bundles of the microtubules are formed, they are stabilized, lose their ability to function normally that causes mitotic inhibition in the cells. Docetaxel is long preserved in the cells, where it reaches its high concentration [2].

The use of docetaxel is connected with numerous and strong side effects (reversible neutropenia, hypersensitivity reaction, the development of the peripheral oedema, sicchasia, vomiting, peripheric neuropathy, heart rhythm disorder, paropsis), representing in some cases hazard to life of the patients. Docetaxel is contraindicated to patients with compromised liver function, as well as pregnant and lactating women.

A major deformity of the anticancer drugs of the taxanes range is the impossibility of their peroral use because of their worse absorption from the alimentary canal [3], in consequence of which the drug must be taken parenterally.

Oral administration of taxanes can substantially reduce toxic side effects in many cases, in comparison with the nowadays used endovenous medical maintenance [4]. In comparison with the quick emergence of high concentrations in the blood, which takes place in the case of endovenous infusion, the absorption of the active agent through the intestinal wall (to which contributes the boosting agent) provides a gradual achievement of therapeutic blood levels and stable maintenance of it during a long period of time. However, as the data of the animal studies show, the use of the peroral dosage forms of taxanes cannot fully solve the problems connected with the toxic property of the drugs and the presence of the numerous side effects (though they partially reduce them).

One solution might be to incorporate docetaxel into liposomes, and there indeed are various publications in literature about the incorporation of docetaxel into liposomes [5, 6, 7, 8] and polymeric submicron elements [5, 9, 10].

The work [10] is of special interest; it describes obtaining a concentrated product of PLGA, surface modifiers and oxidation retarders (vitamin E) in dimethyl sulfoxide (DMSO). These concentrates show sufficient storage stability. Before use, they are mixed with saline solution, and a fine suspension is obtained, suitable for the infusion intake by the patients. The concentration of the DMSO in preparation for infusion does not exceed 3% and does not give significant toxic effects. This drug is not inferior to other dosage forms of activity of docetaxel in the experiments in vitro and in vivo with laboratory animals, and it shows a visible extended effect. It is stated that such concentrates are obtained simply and with high productivity.

The disadvantage in the technology of obtaining such particles by classical methods is the use of a large excess of a surface modifier (serum albumin, SAS) during the process of their synthesis. The high content of surface modifier in the dosage form is in most cases undesirable, because it leads to the reduction of the drug percentage and the deterioration of its physical and chemical properties. To eliminate this unwanted effect nowadays polymer carriers are provided which have “ligated” to them surface modifiers with a covalent bond in the ratio of 1:1. This method of nanoparticles stabilization has been successfully used previously for obtaining liposomes [7]. Use of the diblock copolymers substantially facilitates obtaining of the submicron elements with the drug substance, the obtained elements being notable for stability, good resuspendability and high antineoplastic activity [11, 12, 13].

In in vivo experiments with BALB mice without thymus with vaccinated neoformation of the prostate, docetaxel, included in the elements, showed more efficiency in comparison with the original (free) docetaxel. The development of the neoformation with the laboratory animals was substantially hindered (nearly 2 times) and their life prolongated. In in vivo and in vitro experiments the prolonged effect of the action of drugs submicron forms based on docetaxel emerged noticeably, and also some reduction of toxicity and the demonstration of side effects due to the passive transportation of the medicinal substance were found.

The next stage in the development of technology allowing for obtaining submicron particles on the basis of anti-tumor agents is creating of the systems of directional delivering chemotherapeutic agents to target cells, using vector molecules conjugated with the polymer through the modifying agent's molecule.

In 2006, proceedings of the U.S. Academy of Sciences published a work [13] that had represented a system of the directional delivering the docetaxel anti-tumor agent based on polymeric nanoparticles to prostate cancer cells.

With the development of the molecular cloning methods, a wider opportunity appeared to obtain gene-engineered (or humanized) monoclonal antibodies. However, there is not much progress in this area. It is possible to create systems of targeted delivery on the basis of the polymeric nanoparticles, with the help of the chemical conjugation, or with the superficial persorption of the vector. These vectors have high affinity to the target receptors, and they provide effective endocytosis, non-immunogenic and nonallergenic. In some cases the use of the systems of the targeted delivery on this basis allows to overcome multi-drug resistance of the tumor cells to the chemical drugs. However, their use is reduced by the high price of the final medicinal product.

U.S. Pat. No. 7,981,445 [14] describes a method of obtaining nanoparticles, which is carried out at a ratio of polymer/drug (1:1). Obviously, the amount of PLGA in nanoparticles obtained using said method is very low, and the main micelle-generating composition is serum albumin. The stability of the composition is obtained by adding citrate thereto. The average size of the particles is no greater than about 200 nm.

A rifampicin-based medication with prolonged action was described in RU patent No. 2 418 585 [15], wherein nanoparticles comprising biodegradable polymer of lactic acid or copolymer of lactic acid and glycolic acid, a surface active agent and a cryoprotector were combined with rifampicin.

To solve the problems connected with the toxic properties of the drugs during the treatment of oncologic diseases, it is necessary to meet the problems of the drug efficiency scale-up and, therefore, reduction of the therapeutic dose and toxic effects. The most significant of the possible ways in trying to reach this goal is the design of the drugs or dosage forms of the directional effect, providing the delivery and localization of the drug into the target cells in order to achieve the medical maintenance rates. It is important that this property is associated with the prolonged gradual release of the substance (durability of the action), that allows to reduce the dose of the anticancer drug and to create more comfortable environment for patients who have to use the drug on a regular and long-term basis.

The new technical result is achieved in combination of all the existing signs of the created medicinal agent, on the basis of the docetaxel, allowing to achieve positive pharmacodynamic effect of the drug at smaller rates and thus to lower the risk and the degree of the emergence of the poisonous action, with the simultaneous effect of the action prolongation, reduction of the dosage frequency during the treatment.

Technology for obtaining micro- and nanoscale (submicron) dosage forms with the use of biodegradable polymers can become a promising alternative to conventional pharmaceutical technologies in creating new drugs. Drug substances well-known and commonly used in clinical practice may be included in such dosage forms.

The development of the new dosage forms of the anticancer agents on the basis of the polymeric elements with the targeted delivery is the problem number one at present. While in the above-mentioned publications various solutions have been provided, none of the publications describe the specific nanoparticles disclosed in the present invention.

It follows from the foregoing that at the given stage of the technology obtaining of the submicron polymeric elements with the drug, the traditional ways for obtaining them are still relevant, based on the methods of precipitation, emulsifying and others, with the use of the commercial polymers, SAS and other surface modifiers.

### SUMMARY OF THE INVENTION

The present invention provides a novel docetaxel-based prolonged-release cancer treatment drug which comprises, in stable nanoparticles: docetaxel as an anticancer drug, a polylactic-co-glycolic acid with a 50/50% monomer unit molar ratio (PLGA 50/50) as a biodegradable polymer, serum albumin as a surface-active substance and D-mannitol as a cryoprotective agent, in the following component ratios, in mass percents:

| | |
|---|---|
| Docetaxel | 3.8-4.0 |
| PLGA 50/50 | 38.3-38.7 |
| Serum albumin | 47.5-50.0 |
| D-mannitol | 9.7-9.9. |

### BRIEF DESCRIPTION OF THE DRAWINGS

FIG. 1 Dynamics of death of control animals upon inoculation of mammary adenocarcinoma of the Ca755 (1) line, one-time infusion of free docetaxel (2), and docetaxel as a part of a composition with PLGA 50/50 (Dtx-PLGA 50/50) (3) in the doses of 10 mg/kg within 48 hours after the tumor inoculation into animals.

FIG. 2 Changes in Ca755 mammary adenocarcinoma tumor growth inhibition level in progress upon one-time infusion into animals the free docetaxel (1) and docetaxel as a part of the composition with the PLGA 50/50 (2) polymer in the doses of 10 mg/kg within 48 hours upon the tumor inoculation. The critical level (50%) of the tumor growth inhibition is (3).

### DETAILED DESCRIPTION OF THE INVENTION

The present invention is made in the field of pharmacology and medicine, specifically it relates to a new generation of docetaxel-based prolonged-release antitumor agents. A composite docetaxel-based drug was invented, which has a high specific activity and reduced toxicity, and differs from other solutions of the kind through the fact that it represents bio-digestible stable nanoparticles. The application of the invented drug will allow doctors to reach the desired effects using smaller therapeutic doses and within shorter times, and make the antitumor therapy with the developed drug safer.

Technical result of the present invention is achieved by including into the new pharmacon, on the basis of the docetaxel, commercially affordable biodegradable polymers, accepted to medical application for obtaining nanoparticles. In its capacity copolymer of the glycolic and hydroxy propionic acid (PLGA) is used. Appropriateness of the use of the microsomic drugs for the cancer treatment is determined by the prolongation of the action and the possibility of the actualization of the targeted transport of the drug substance into the neoformation.

To obtain nanoparticle polymers with gram-molecular weight from 10 to 300 kDa and molecular ratio of the remainders of the hydroxypropionic acid and glycolic acid 50:50% were used. To obtain a stable dosage form representing the nanoparticle size 250-500 nm and having a sustained release of docetaxel, surface-active agent is also used: serum albumin and cryoprotectant: D-mannitol. The proposed pharmacon is obtained by the well-known method of the simple (single) emulsifying (water/oil) [5]. Sorption of docetaxel inside nanoparticles of the polymer occurs during the formation of a suspension during removal of the organic solvent from the resulting emulsion.

Thus, on the basis of the experiment made, the optimal conditions and substances are as follows:
1. Polymer used for preparing a composition with docetaxel, is preferably PLGA 50/50;
2. The optimal mass correlation of PLGA 50/50 to docetaxel is equal to 10:1;
3. The best from the examined SAS is 0.5% liquid albumin from the blood serum of a human being, or recombinantly produced human serum albumin;
4. The optimal mode of the emulsifying: 24 thousand rounds per minute, 3 times for 1 minute with 1 minute intervals;
5. The optimal volumetric proportion of the organic and water phases is 1:5.

The developed drug can be manufactured in the form of enterosoluble tablets, capsules, granules, powder, or in any other peroral form on the basis of the well-known technologies.

The method for obtaining nanoparticles as described in reference [14] is carried out at a ratio of polymer/drug (1:1), which is significantly lower than what the present inventors use in obtaining the nanoparticles (10:1). It is obvious that in the product obtained with the method of reference [14] the mass content of the PLGA is very low and the main micelle-forming component is human serum albumin. At the same time, the main structural component of the elements, obtained by the methodology proposed in the present invention is in particular PLGA, and serum albumin is used only as surface modifier of the particle surface and its content in the product is not high.

Consequently, the elements described in the method of said patent fundamentally differ in their structure from those obtained in the present invention. So, the product of said patent may be considered as a remote analogue of the drug obtained with the present method.

According to the results obtained from testing the new anti-tumor docetaxel drug based on the biodegradable polymer PLGA 50/50, as shown in the Examples below, we can conclude with confidence that the docetaxel polymer composition of the present invention has a higher specific activity and expressed prolonging effect at practically the same toxicity, as compared to the free docetaxel. Such results allow us to draw the conclusions on the growth of safety margins and the increase in the therapeutic index at simultaneous reducing the dosage frequency of the new drug. It is also important to note that including docetaxel into the polymeric matrix contributes to more efficient passing the barriers causing the resistance of tumor cells against drugs used for therapy.

The present invention is illustrated by the following Examples.

### Example 1. Obtaining of the Polymeric Nanoparticles with Incorporated Docetaxel into them by the Method of Simple Emulsion

A mixture made up of the solution of 20-40% of the biodegradable polymer (PLGA) and 1-10% docetaxel in an organic vehicle (dichloromethane) and also 0.5-1% liquor polymeric emulsion stabilizer—SAS (serum albumin) in the volumetric proportion to the organic vehicle 5:1 is homogenized during 24 thousand rounds per minute with the help of the tissue grinder Ultra-Turrax® T-25 (IKA®, Federal Republic of Germany) 3 times in 1 minute with 1 minute intervals. The emulsion is stirred for 1 hour to complete removal of the organic solvent. This work should be carried out during the operation of the local exhaust ventilation. The resulting suspension is filtered through a glass porous filter (size of the pores is 40-110 micrones), is added into infiltration 1-3% (volume) of the cryoprotectant (D-mannitol), is frozen and lyophilized. The average size of the elements, defined by the method of the photon correlative spectroscopy at the submicron laser spectrometer Coulter N4MD (USA), is from 250 to 500 nm, promoting the effective absorption in the gastrointestinal tract. The size of the obtained elements depends on the type of polymer, emulsion stabilizers, their concentrations, as well as on the conditions of the homogenization. The degree of the docetaxel integration into the polymer elements ranges from 15 to 70%.

| The optimal composition | mass % |
|---|---|
| Polymer (PLGA 50/50) | 38.3-38.7 |
| Docetaxel | 3.8-4.0 |
| Serum albumin | 47.5-50.0 |
| Cryoprotectant (D-mannitol) | 9.7-9.9 |
| The size of the elements | 330-360 nm |

### Example 2. Comparative Pharmacological Efficacy of the Docetaxel-Based Prolonged-Release Drug and Free Docetaxel

To investigate the antitumor activity of a new dosage form of docetaxel in the composition with PLGA 50/50 the acinic cell carcinoma of the glandula mammaria of the NZB mouse Ca 755 was chosen. As the comparison object free docetaxel substance was used. The experiments were carried out on C₅₇B1/6 female mice with the body weight of 18-20 g.

The anti-tumor activity of the preparations under research was estimated on the basis of the comparative analysis of tumor growth kinetics and comparing the lifetime in test groups and control groups of animals.

The introduction of both the docetaxel-based prolonged-release drug and the free docetaxel was started 48 hours after tumor inoculation.

The docetaxel polymeric composition and the docetaxel substance were both proved to have a strong inhibiting effect upon the tumor growth, after being introduced into the mammary-adenocarcinoma-inoculated laboratory animals. The efficiency of the polymeric nanosomal form of the preparation exceeds significantly the activity of the equivalently dosed free substance.

After introducing the docetaxel nanosomal preparation, like in case of the docetaxel substance, the tumor growth inhibiting effect starts to occur in 7-8 days, the effect of the composition preparation being more striking. The difference between the effects of the docetaxel polymeric composition and the docetaxel substance had increased by the 12^{th} day upon introducing thereof into the animals and retained within the entire experiment period (23 days) for all doses used. The tumor growth inhibition depends on the dose of the preparation introduced, i.e. it is of the dose-depending nature, for both the substance and the preparation in form of polymeric nanoparticles.

The essential effect of the drug (tumor growth inhibition above 50%) as particles is retained for over 23 days when introduced in the doses of 10 or 20 mg/kg, while for the docetaxel substance this period does not exceed 17 days even for the dose of 20 mg/kg. Data listed above indicates a significantly higher anti-tumor activity of the new drug and the availability of prolonged release, as compared to the docetaxel substance.

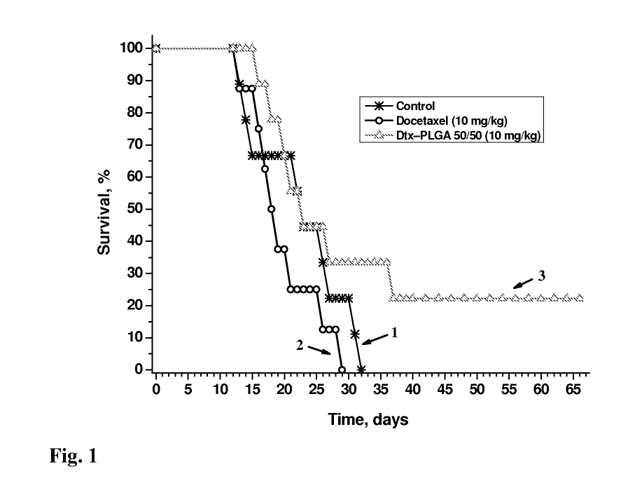

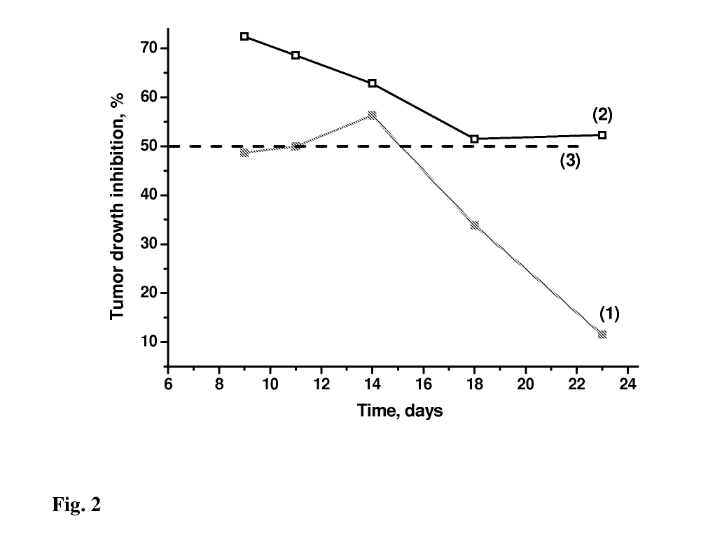

It should be noted that the nanosomal drug in the dose of 20 mg/kg demonstrated lower efficiency than in the dose of 10 mg/kg 19 days after introduction. Particularly, the positive results should be noted, obtained from treating the tumor-inoculated mice with the polymer composition of docetaxel for the dose of 10 mg/kg (FIG. 1 and FIG. 2) (the animals survived even 2 months upon tumor inoculation). Applying the docetaxel polymeric composition in the dose of 20 mg/kg did not contribute to the animal survival rate, which, seemingly, is determined by the toxic effects of the drug.

Treating tumor-inoculated mice with the nanosomal drug in compliance with a pre-set schedule allows tumor growth inhibiting and increasing the lifetime, as compared with free docetaxel, reducing, at the same time, the dosage frequency due to the prolonged release of the new drug developed.

### Example 3. Toxicological Features of the Docetaxel-Based Prolonged-Release Original Drug

To estimate the degree of toxic reactions to the introduction of drugs, the in-progression peripheral white blood count was studied during and upon completion of the drug infusions.

The mouse white blood cell count was performed in the relevant groups of mice in 3 days after the experiment was started. The white blood cells were counted in the Gorjaev's chamber after 10 μl of blood taken from caudal vein had been dissolved in 40 μl of the 3% solution of acetic acid. When used in equivalent doses (5 and 10 mg/kg), Docetaxel being a part of the composition with PLGA 50/50 was proved to have higher toxicity (on the fourth day upon the Ca755 mammary adenocarcinoma inoculation and on the 3^{rd} day upon the drug introduction) than free docetaxel, though this affect was not great as compared with the control group.

Toxic effect upon the white blood levels, provided by both medicines used in the dose of 20 mg/kg is practically the same, and it significantly increased as compared to the control group.

### REFERENCES

1. RLS-Entsiklopedia lekarstv® [Encyclopaedia of Medicines] [In the Russian language]. Izd. 8-ye pererab. i dop./G1. red. Yu. F. Krylov. M. “RLS-2001”, 2000. P. 321-322.

2. Kovtun V. A., Gayevoy K. V., Sevidov V. V. Dlitelnaya infuziya dotsetaksela (Taksotera) v polikhimioterapii 2-y linii u bolnykh s progressiruyushchimi solidnymi opukholyami. [Long-Lasting Docetaxel (Taxotere) Infusion in Polychemotherapy in Patients Suffering from Accelerating Solid Tumors] [In the Russian language].//Onkologiya 2006. P. 8. No. 3. P. 285-286.

3. Spravochnik Vidal 2010. Opisaniye lekarstvennogo preparata TAXOTERE® [Vidal Drug Information System. Description of TAXOTERE®] [In the Russian language].

4. Severin Ye. S., Rodina A. V. Problemy i perspektivy sovremennoy protivoopukholevoy terapii. [Challenges and Propects of the Modern Anti-Tumor Therapy] [In the Russian language]. //Uspekhi biol. khimii 2006. V. 46. P. 43-64.

5. Musumeci T., Ventura C. A., Giannone I. et al. PLA/PLGA nanoparticles for sustained release of docetaxel.//Int. J. Pharm. 2006. V. 325 (1-2). P. 172-179.

6. Immordino M. L. et al. Preparation, characterization, cytotoxicity and pharmacokinetics of liposomes containing docetaxel.//J. Control. Release 2003. V. 91 (3). P. 417-429.

7. Yousefi A. et al. Preparation and In Vitro Evaluation of a Pegylated Nano-Liposomal Formulation Containing Docetaxel.//Sci. Pharm. 2009. V. 77 (2). P. 453-464;

8. Zhai G. et al. Preparation, characterization and pharmacokinetics of folate receptor-targeted liposomes for docetaxel delivery.//J. Nanosci. Nanotechnol. 2009. V. 9 (3). P. 2155-2161;

9. Feng Si. S., Mei L. et al. Poly(lactide)-Vitamin E derivative/montmorillonite nanoparticle formulations for the oral delivery of Docetaxel.//Biomaterials 2009. V. 30 (19). P. 3297-3306;

10. U.S. Pat. No. 2,370,261 C2 PΦ. Mπκ A61K 31/337, A61K 47/20, A61K 47/34, A61K 9/107, A61P 35/00. Published on Oct. 20, 2009. Stabilnaya emulsiya dlya parenteralnogo vvedeniya plokho rastvorimykh v vode soyedineniy, obladayushchikh protivoopukholevoy aktivnostyu, i sposobov yeyo polucheniya [Stable Emulsion for Parenteral Introduction of Poorly Water-Soluble Compounds Having Anti-Tumor Properties, and the Method of Producing Thereof] [In the Russian language]./Klyachko N. L., Uglanova S. V., Abrikosova Yu. Ye. et al.

11. Ling Y, Huang Y Preparation and Release Efficiency of Poly(lactic-co-glycolic)Acid Nanoparticles for Drug Loaded Paclitaxel./Y Peng, X. Weng (Eds.): APCMBE 2008, IFMBE Proceedings 19, P. 514-517;

12. Cheng J. et al. Formulation of functionalized PLGA-PEG Nanoparticles for in vivo targeted drug delivery.//Biomaterials 2007. V. 28 (5). P. 869-876;

13. Farokhzad O. C. et al. Targeted nanoparticle-aptamer bioconjugates for cancer chemotherapy in vivo. II PNAS 2006. V. 103 (16). P. 6315-6320;

14. U.S. Pat. No. 7,981,445 B2 USA (2011). Compositions and methods for preparation of poorly water soluble drugs with increased stability./De T., Desal N. P., Yang A. et al.

15. RU patent No. 2418585 (publ. 20.5.2011) Rifampicin-based medication with prolonged action for treatment of resistant forms of tuberculosis.//Severin, E. et al.

1. A docetaxel-based prolonged-release cancer treatment drug, comprising in stable nanoparticles:
a) docetaxel,
b) a polylactic-co-glycolic acid with a 50/50% monomer units molar ratio (PLGA 50/50) as a biodegradable polymer,
c) serum albumin as a surface-active substance and
d) D-mannitol as a cryoprotective agent, in the following component ratios, in mass percents:

| | |
|---|---|
| Docetaxel | 3.8-4.0 |
| PLGA 50/50 | 38.3-38.7 |
| Serum albumin | 47.5-50.0 |
| D-mannitol | 9.7-9.9. |

2. The drug according to claim 1, wherein the serum albumin is obtained from human blood serum.

3. The drug according to claim 1, wherein the serum albumin is recombinant human serum albumin.

4. The drug according to claim 1, wherein the nanoparticles are 250 to 500 nm in size.

5. The drug according to claim 1, which is manufactured in peroral form.

6. The drug according to claim 5, wherein the peroral form is the form of tablets, capsules, granules or powder.
